(19) United States
(12) Patent Application Publication
(10) Pub. No.: US 2026/0056544 A1
(43) Pub. Date: Feb. 26, 2026

(54) UNMANNED AERIAL SYSTEM (UAS) WITH VISION ALGORITHM PACKAGE FOR MEDICAL SITUATION AWARENESS

(71) Applicant: The Government of the United States, as Represented by the Director of the Defense Health Agency, Fort Detrick, MD (US)

(72) Inventors: Ethan Thomas Quist, Frederick, MD (US); Nathan Fisher, Frederick, MD (US); Kelly Nicole Roman, Falls Church, MD (US); Justin Halliday Peel, Falls Church, MD (US)

(21) Appl. No.: 19/306,905

(22) Filed: Aug. 21, 2025

Related U.S. Application Data
(60) Provisional application No. 63/686,427, filed on Aug. 23, 2024.

(51) Int. Cl. G05D 1/224; B64U 10/80; B64U 20/87; B64U 101/30; B64U 101/55; G06V 20/17; G06V 40/10; G16H 40/67

(52) U.S. Cl. CPC G05D 1/2244; B64U 10/80; B64U 20/87; G06V 20/17; G06V 40/103; G16H 40/67; B64U 2101/30; B64U 2101/55

(57) ABSTRACT

A system for providing medical situational awareness in a hazardous area includes an unmanned aerial system (UAS) and a UAS controller for operating the UAS. The UAS includes at least one electro-optical (EO) sensor for viewing the hazardous area to provide EO sensor data, and a processor for executing a vision algorithm package based on the EO sensor data to locate and analyze casualty victims within the hazardous area. The vision algorithm package includes casualty detection algorithms for casualty detection and identification; casualty assessment algorithms for casualty respiration rate detection, pulse rate detection and motion detection; and gross injury detection algorithms for casualty wound and hemorrhage detection, and kinematic irregularity detection. Casualty information as determined by the vision algorithm package is transmitted to the UAS controller. The UAS controller includes a display to display the casualty information to provide the medical situation for the hazardous area.

### RELATED APPLICATION

The present application claims the benefit of U.S. Provisional Patent Application Ser. No. 63/686,427 filed Aug. 23, 2024, all of which is fully incorporated by reference.

### TECHNICAL FIELD

The present disclosure relates to unmanned aerial systems (UAS), and, more particularly, to a UAS with a vision algorithm package for providing medical situational awareness to a medic in a hazardous area.

### BACKGROUND

Locating and assessing casualties in a hazardous area, such as a battlefield, involves significant risk to life and limb for other soldiers, reducing the readiness of a fighting force. Soldiers/medics teaming with an unmanned aerial system (UAS) during casualty extraction missions may offload certain tasks, reducing the medic's cognitive and physical burden and expediting the detection and assessment of casualties while providing standoff protection. Nonetheless, there is still a need to improve locating and assessing casualties in a hazardous area.

### SUMMARY

A system for providing medical situational awareness in a hazardous area includes an unmanned aerial system (UAS) and a UAS controller for operating the UAS. The UAS includes a housing, at least one electro-optical (EO) sensor carried by the housing and configured for viewing the hazardous area to provide EO sensor data. A processor is carried by the housing and is coupled to the at least one EO sensor, and is configured to execute a vision algorithm package based on the EO sensor data to locate and analyze casualty victims within the hazardous area.

The vision algorithm package includes casualty detection algorithms for casualty detection and identification; casualty assessment algorithms for casualty respiration rate detection, pulse rate detection and motion detection; and gross injury detection algorithms for casualty wound and hemorrhage detection, and kinematic irregularity detection. A transceiver is carried by the housing and is coupled to the processor and configured to receive control signals and to transmit live casualty information as determined by the vision algorithm package. The UAS controller is for providing the control signals to the UAS, and includes a display to display operator data for the UAS and the live casualty information received from the transceiver to provide the medical situation awareness for the hazardous area.

The UAS controller includes a processor to aggregate the live casualty information over time, with the aggregated casualty information being transmitted to a network based on a confidence factor of the aggregated casualty information.

The system includes at least one mobile device in proximity to the casualty victims and is configured to receive and display the aggregated casualty information from the network.

The vision algorithm package provides the casualty information in real-time to the UAS controller.

The casualty detection algorithms includes a human detection algorithm, and a human identification algorithm. The human detection algorithm is configured to detect individual body parts of casualty victims when partially occluded within the hazardous area.

The casualty assessment algorithms include a respiration rate detection algorithm, a pulse rate detection algorithm and a motion detection algorithm.

The gross injury detection algorithms include a wound and hemorrhage detection algorithm, and a kinematic irregularity detection algorithm.

The UAS may be configured as a small UAS (SUAS) weighing less than 55 pounds. The at least one EO sensor may include a camera.

The UAS includes a global positioning system (GPS) to determine GPS coordinates of the casualty victims based on an angle trajectory of the camera.

Another aspect is directed to a method for providing medical situational awareness in a hazardous area using the system as described above. The method includes viewing the hazardous area to provide EO sensor data, and executing a vision algorithm package based on the EO sensor data to locate and analyze casualty victims within the hazardous area. The vision algorithm package includes casualty detection algorithms for casualty detection and identification; casualty assessment algorithms for casualty respiration rate detection, pulse rate detection and motion detection; and gross injury detection algorithms for casualty wound and hemorrhage detection, and kinematic irregularity detection. Control signals are received for control of the UAS. The casualty information as determined by the vision algorithm package is transmitted to the UAS controller. The UAS controller is used for providing the control signals to operate the UAS, and to display operator data for the UAS and to display the live casualty information received from the UAS to provide the medical situation awareness for the hazardous area.

### BRIEF DESCRIPTION OF THE DRAWINGS

FIG. 1 is a schematic diagram of a system for providing medical situational awareness in a hazardous area in which various aspects of the disclosure may be implemented.

FIG. 2 is a processing diagram of algorithms in the vision algorithm package illustrated in FIG. 1.

FIG. 3 is a graphical representation illustrating operation of the system illustrated in FIG. 1.

FIG. 4 is a graphical representation of an initial casualty assessment through pulse, respiration, and movement detection for the system illustrated in FIG. 1.

FIG. 5 is a graphical representation of body part segmentation for the system illustrated in FIG. 1.

FIG. 6 is a graphical representation of wound detection for the system illustrated in FIG. 1.

FIGS. 7-8 are partial screen shots of a casualty as viewed on the UAS controller with and without zoom as provided by the UAS illustrated in FIG. 1.

FIGS. 9-11 are full screen shots of a casualty as viewed on the UAS controller as provided by the UAS illustrated in FIG. 1.

### DETAILED DESCRIPTION

The present description is made with reference to the accompanying drawings, in which exemplary embodiments are shown. However, many different embodiments may be used, and thus the description should not be construed as limited to the particular embodiments set forth herein. Rather, these embodiments are provided so that this disclosure will be thorough and complete. Like numbers refer to like elements throughout.

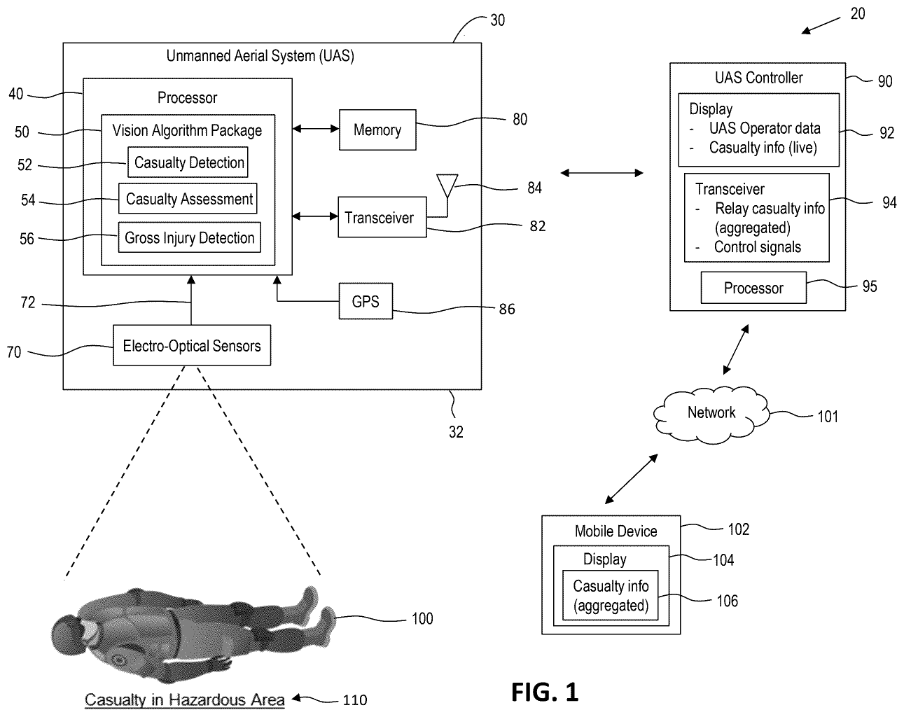

Referring initially to FIG. 1, a system 20 for providing medical situational awareness in a hazardous area 110 will be discussed. The hazardous area 110 may be a battlefield, for example.

In a battlefield environment, it is expected that operations will cover geographically dispersed areas, complex terrain, and high threat conditions. Units operating in these conditions may not have full line of sight or complete positional awareness of deployed troops when injured.

Survivability of combat casualties may be maximized by quickly delivering the appropriate medical interventions. Delay of care may significantly increase rates of both complications and eventual mortality in patients with trauma injuries leading to suboptimal outcomes. This critical period for care may be called the “golden hour.” The system 20 advantageously provides medical situation awareness for the hazardous area 110 to facilitate rapid planning and action by a medic.

The system 20 includes an unmanned aerial system (UAS) 30 and a UAS controller 90 for operating the UAS 30. The UAS 30 may also be referred to as a small unmanned aerial system (SUAS), which is a remotely piloted drone that weighs less than 55 pounds. The UAS 30 may further be referred to as a drone.

The UAS 30 includes a housing 32, and at least one electro-optical (EO) sensor 70 carried by the housing 32. The EO sensor is configured as a camera for viewing the hazardous area 110 to provide EO sensor data. A processor 40 and a memory 80 coupled to the processor 40 are carried by the housing 32. The processor 40 is coupled to the EO sensor 70, and executes a vision algorithm package 50 based on the EO sensor data to locate and analyze casualty victims 100 within the hazardous area 110.

The vision algorithm package 50 includes casualty detection algorithms 52 for casualty detection and identification, casualty assessment algorithms 54 for casualty respiration rate detection, pulse rate detection and motion detection, and gross injury detection algorithms 56 for casualty wound and hemorrhage detection, and kinematic irregularity detection. A breakdown of these detection algorithms will be provided below.

The UAS 30 includes a transceiver 82 and an antenna 84 coupled to the transceiver, both of which are carried by the housing 32. The transceiver 82 may operate, for example, at Wi-Fi frequencies, such as 2.4 GHz or 5 GHZ. These operating frequencies are not to be limiting. The transceiver 82 may be configured to operate at other frequences to support the intended operating environment.

The transceiver 82 receives control signals for control of the UAS 30 and transmits live casualty information as determined by the vision algorithm package 50 to the UAS controller 90. The UAS controller 90 provides the control signals to the UAS 30, and includes a display 92 to display operator data for the UAS 30 and the live casualty information to provide the medical situation awareness for the hazardous area 110.

The UAS 30 needs to hover with a clear view over a casualty 100 long enough to collect vitals. The UAS controller 90 receives a live stream from the UAS 30. This may be 30 seconds or longer. A processor 95 within the UAS controller 90 evaluates the vitals of the casualty 100. When a confidence threshold is reached, then the processor 95 aggregates or summarizes the live casualty information over time, with the aggregated casualty information then being transmitted to a network 101. The live casualty information over time is aggregated before being pushed or transmitted to the network 101 so as to not overload the network.

The system 20 includes at least one mobile device 102 in proximity to the casualty victims 100 and is configured to receive and display the aggregated casualty information from the network. The displayed aggregated casualty information includes a map showing location of the casualties 100 found and their vital signs. There may be multiple medics in the hazardous area 110, with each medic receiving the aggregated casualty information on their mobile device 102.

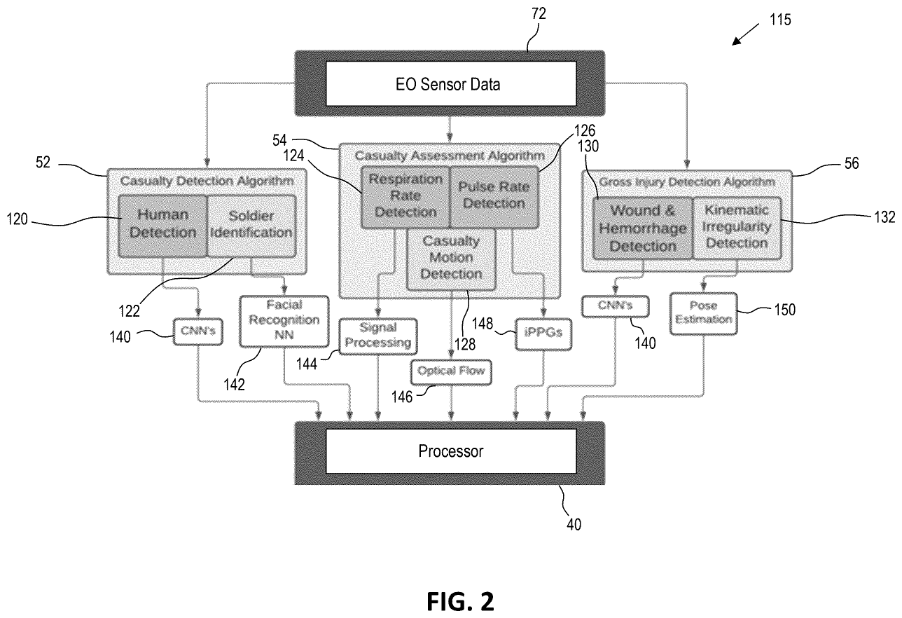

Referring now to FIG. 2, a processing diagram 115 of algorithms in the vision algorithm package 50 will be discussed. The algorithms 52, 54 and 56 operate in response to EO sensor data 72 received from the electro-optical sensors 70.

The casualty detection algorithms 52 include a human detection algorithm 120, and a human (e.g., soldier) identification algorithm 122. The casualty assessment algorithms 54 include a respiration rate detection algorithm 124, a pulse rate detection algorithm 126 and a casualty motion detection algorithm 128. The gross injury detection algorithms 56 include a wound and hemorrhage detection algorithm 130, and a kinematic irregularity detection algorithm 132.

A convolutional neural network (CNN) 140 is used by the human detection algorithm 120 and wound and hemorrhage detection algorithm 130. A CNN 140 is a feed-forward neural network that learns by itself via filter optimization. A facial recognition neural network (NN) 142 is used by the human identification algorithm 122.

Image photoplethysmography (iPPG) 148 is used by the pulse rate detection algorithm 126. This is a technique for remote non-contact pulse rate measurement. iPPG 148 is usually acquired from facial or palm video. This package provides tools for iPPG signal extraction and processing. Optical flow 146 is used by the casualty motion detection algorithm 128. Optical flow is the pattern of apparent motion of image objects between two consecutive frames caused by the movement of the object or the camera. It is 2D vector field where each vector is a displacement vector showing the movement of points from first frame to second.

Signal processing is used by the respiration rate detection algorithm 124. Pose estimation 150 is used by the kinematic irregularity detection algorithm 132. Pose estimation 150 is the task of using an ML model to estimate the pose of a person from an image or a video by estimating the spatial locations of key body joints (key points).

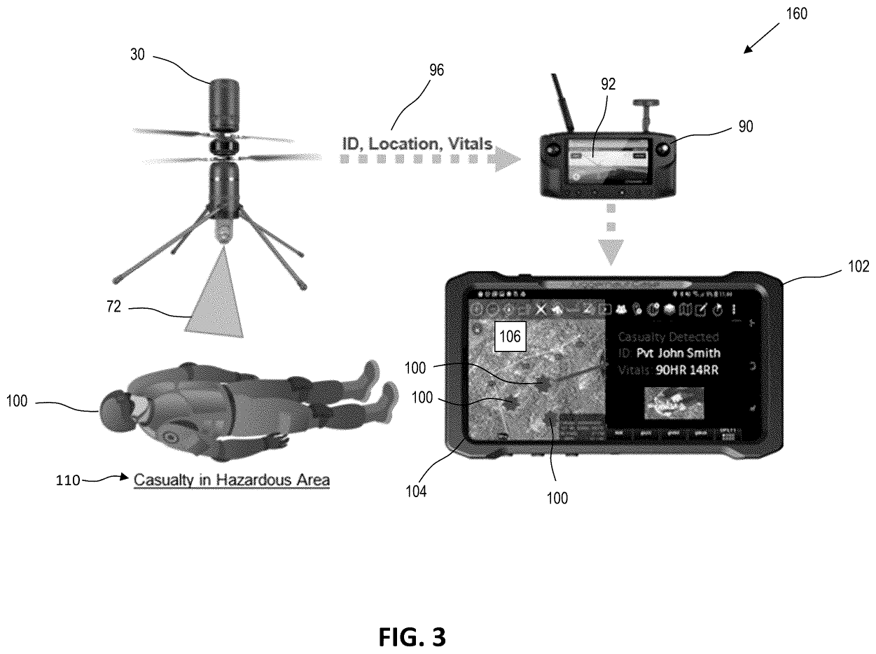

A graphical representation 160 illustrating operation of the system 30 will be discussed in reference to FIG. 3. The UAS 30 collects EO sensor data 72 on a casualty 100 in a hazardous area 110. The vision algorithm package 50 within the UAS 30 performs image processing to interpret the EO sensor data 72 to provide casualty information 96 to the UAS controller 90. The casualty information 96 includes, for example, identification, location and vitals of the casualty 100. This is displayed in real-time.

The display 92 on the UAS controller 90 thus provides a medical situation awareness in real-time. This may be viewed by a medic if the medic is in proximity to the UAS operator. Otherwise, if the UAS operator is remotely located, then the aggregated casualty information over time is pushed or transmitted to the network 101 to be received on the mobile device 102 carried by the medic in proximity to the casualty 100.

The network 101 may be an Android Tactical Assault Kit (ATAK) network, which is a geospatial mapping and situational awareness application designed for Android devices. It is used by military, first responders, and even outdoor enthusiasts for real-time communication, collaboration, and information sharing. ATAK provides a comprehensive view of a user's surroundings, including the ability to share locations, track personnel, and overlay various data layers onto maps. The system 20 is not limited to the (ATAK) network, as other networks may be readily used.

The aggregated casualty information 106 displayed on the mobile device 102 shows that a casualty 100 has been detected. A map on location of the casualty 100 is displayed. Also displayed is the identification and vitals of the casualty 100, and a zoomed in image of the casualty 100 may be provided. More than one casualty 100 may be displayed at a time.

The small size and advanced maneuverability of the UAS 30 is useful as a reconnaissance tool, affording quick and efficient scans of large distances while keeping a small detection profile and low sound footprint. The EO sensor 70 support various missions, and operation of the UAS 30 on the battlefield extends its capabilities to autonomously detect, identify, and remotely assess combat casualties. The vision algorithm package 50 performs image processing to interpret the EO sensor data to provide the casualty information 96 relevant for the medical situation awareness 94.

Vision-based human detection and identification detection has been an active area of research in the artificial intelligence and machine learning (AI/ML) communities. Specifically, the tools developed through deep learning have created models that accurately, and in real-time, detect human subjects in an image or video feed.

Commercially available models for this task exist, yet, like all deep learning models, are inherently biased towards the data they were trained on. State-of-the art models trained on large civilian datasets thus often fail in many combat casualty scenarios. Testing shows that these models fail to detect people when they are heavily obscured, in camouflage, in odd poses, or even just laying down. This includes lying down prone, lying on its side, lying under rubble, or in any awkward position. Though research continues to improve these algorithms for civilian detection and pose mapping, the lack of appropriate data for military casualty detection continues to limit their use for combat casualty applications.

Techniques like vision-based facial recognition and nametag recognition will allow the UAS 30 to identify found causalities. This information is critical in planning and preparing for casualty extraction.

The casualty detection algorithm 52 addresses the lack of relevant data through augmentation, simulation, and the collection of data in realistic combat casualty environments. This, combined with a network architecture specifically designed for casualty discovery, has allowed the development of the casualty detection algorithm 52 to successfully perform in representative field tests by positively detecting and identifying humans.

An appropriate EO sensor (i.e., camera) 70 is used to provide accurate detection from a long standoff distance with a wide field of view without overloading the processor 40. These detection algorithms are developed with deep neural networks (DNN), specifically convolutional neural network (CNN) frameworks which have been proven to be effective for image processing and object classification. Identification algorithms will consist of friendly uniform classification, nametag detection, and face recognition (FR) through CNNs.

The identification detection algorithm 122 will first look to the uniform and nametape (nametag) of the casualty 100 to identify that it is friendly and then to determine the name of the casualty. Both uniform and nametape classification algorithms are similarly developed through deep learning using CNNs. Clothing has already been proven to be classified for forensic purposes using CNNs and this will focus the training sets to friendly combat uniforms.

With the established ability of deep learning architectures to read text, this algorithm refines the capability to apply it to the specific text found on the nametape of a uniform. In order to train the FR DNNs, publicly available datasets such as surveillance cameras face database (SCface) contain thousands of images. With the FR architecture developed it will then be trained on a smaller and focused dataset of faces that would represent a platoon. Using these multiple methods increases the probability of correctly identifying friendly casualties when either the soldier's face or nametape are obscured or occluded in the image.

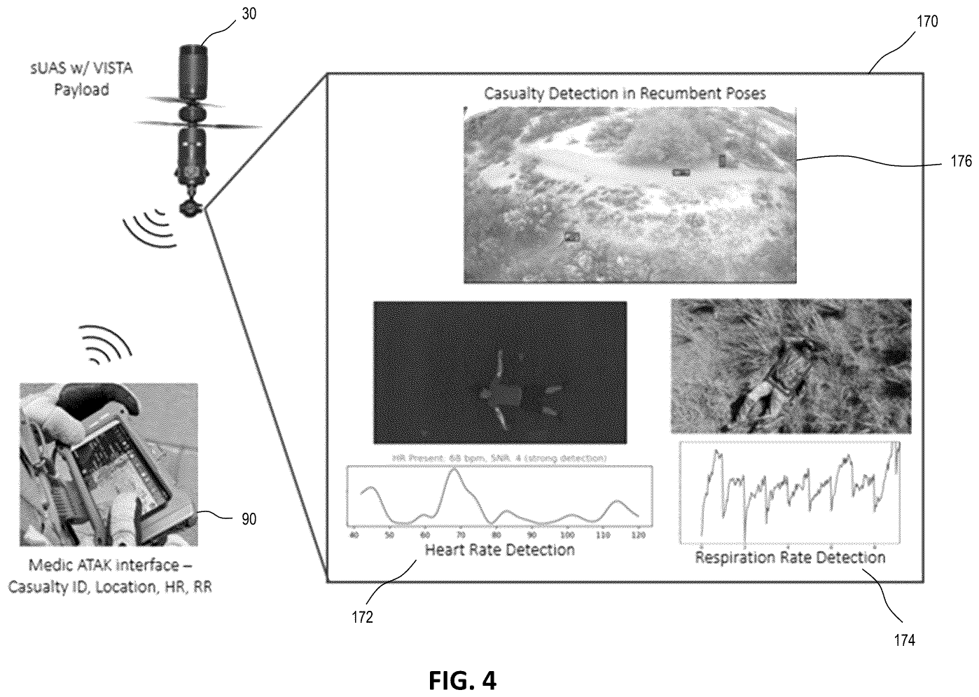

Referring now to FIG. 4, a graphical representation 170 of a vision-based initial casualty assessment through pulse, respiration, and movement detection will be discussed. Once a casualty is identified by the UAS 30, a rapid assessment of the casualty's condition is needed. This condition can be assessed remotely by measuring vital signs, including heart rate (i.e., pulse) detection 172 and respiration rate detection 174, through video using well established techniques. Pulse is measured through imaging photoplethysmography (iPPG), which detects the pulse through minor changes in skin color, and respiration is detected by measuring small motions involved in breathing.

Both techniques require a considerable amount of signal processing to extract the weak signals from noisy imagery. Noise suppression and weak signal estimation are used to produce robust algorithms that can perform under chaotic and dynamic combat casualty environments. Casualty detection in recumbent pose 176 is provided by the kinematic irregularity detection algorithm 132.

The primary challenge in extending these algorithms into natural environments is the difficulty in determining which pixels are signal-bearing, and which contain only noise. By leveraging a custom-designed human detection algorithm 120, which accurately segments the imagery, the vitals signal can be better filtered to improve this measurement in relevant environments. The ability to detect vitals in difficult situations, such as low-lighting conditions and poor image resolution is critical.

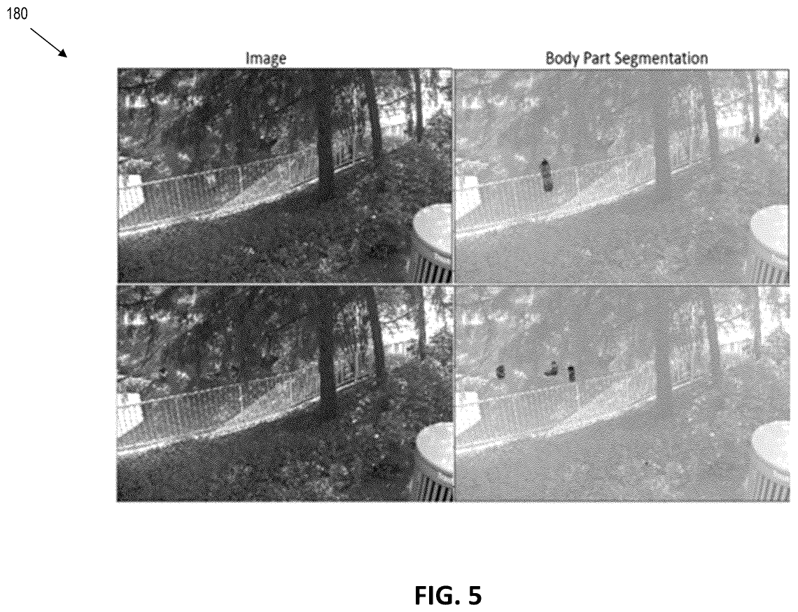

Referring now to FIG. 5, a graphical representation 180 of body part segmentation will be discussed. In many casualty situations, such as explosive blasts, there is a high likelihood of debris and rubble at the scene. Dirt covering the skin and extensive rubble occluding the casualties may provide obstacles to determining the status of the casualties.

The human detection algorithm 120 uses body part segmentation to identify limbs and body parts of a casualty even though the casualty may be partially occluded or in odd poses. The body part segmentation is depicted in different colors.

In addition to determining pulse rate and respiration rate, a casualty movement detection algorithm 128 is used to determine that a casualty is alive and conscious through even the slightest of movements.

As noted above, detecting pulse rate (PR) and respiration rate (RR), and inferring alive/dead and conscious/unconscious states, from a large standoff distance imposes challenging requirements on image resolution and stability. Consequently, stabilization algorithms are executed within the UAS 30.

These stabilization algorithms allow for steady data acquisition in the region of interest around a detected casualty. Pulse rate and respiration rate algorithms do not typically rely on DNNs but instead require more specialized computer vision techniques, such as imaging photoplethysmography, optical flow and spectral analysis.

In casualty search scenarios such as sudden blast trauma there is an expectation that immense dirt on the skin and rubble can interfere with both the pulse rate and respiration rate detection algorithms. A motion detection algorithm will determine a casualty's status when the other algorithms are inhibited. The motion detection algorithm has the capability of determining basic levels of consciousness and status of a casualty from a far standoff distance regardless of occlusions on the body. The algorithm can leverage optical flow to determine casualty movements regardless of the motion of the UAS 30 itself. With the motion detection algorithm in operation, it will cooperate with existing vitals sign algorithm for pulse rate and respiration rate.

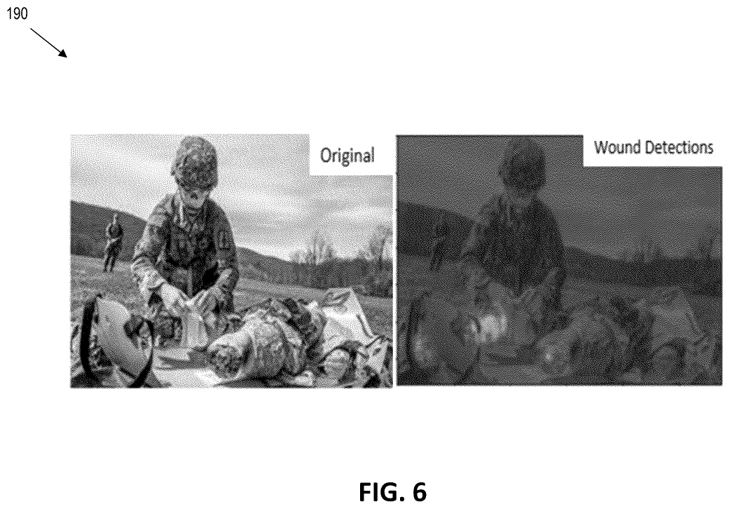

Referring now to FIG. 6 is a graphical representation 190 of wound detection will be discussed. Wound detection is another critical capability for remotely assessing combat casualties and providing actionable information to first responders as quickly as possible. In particular, the wound and hemorrhage detection algorithm 130 and the kinematic irregularity detection algorithm 132 advantageously provide another major piece of casualty assessment information by having the ability to spot kinematic irregularities based on pose analysis.

These detections are critical for triaging and handling casualties in preparing for medical care and extraction. Kinematic irregularities such as broken bones and amputations may be detected through post-processing steps which will weigh in pose estimation information, such as anatomy detection confidence percentages. Furthermore, wounds may be identified, such as burns and bleeding.

To achieve a vision system capable of identifying and classifying major kinematic irregularity injuries such as broken bones and amputations, CNNs are used for wound classification and pose estimation frameworks. The wound detection neural networks typically use masks to isolate regions of interest to help determine if a wound is present. These classifiers are augmented with information from pose estimation and key point anatomy algorithms provide skeletal position information on the wounds. This allows a broken tibia or a transfemoral amputation to be correctly labeled, for example.

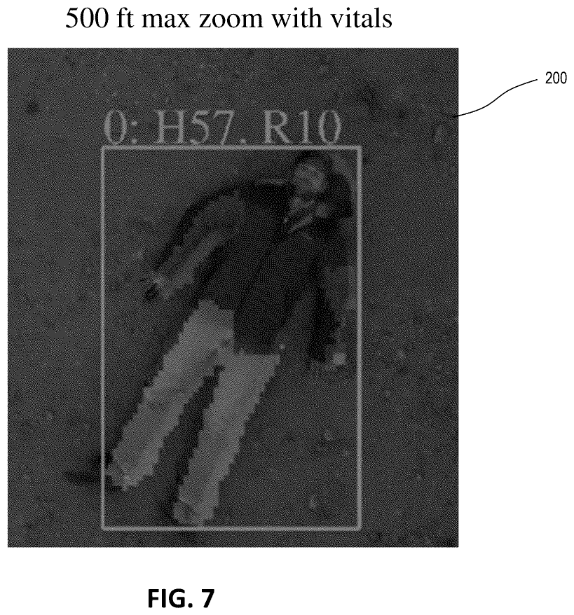

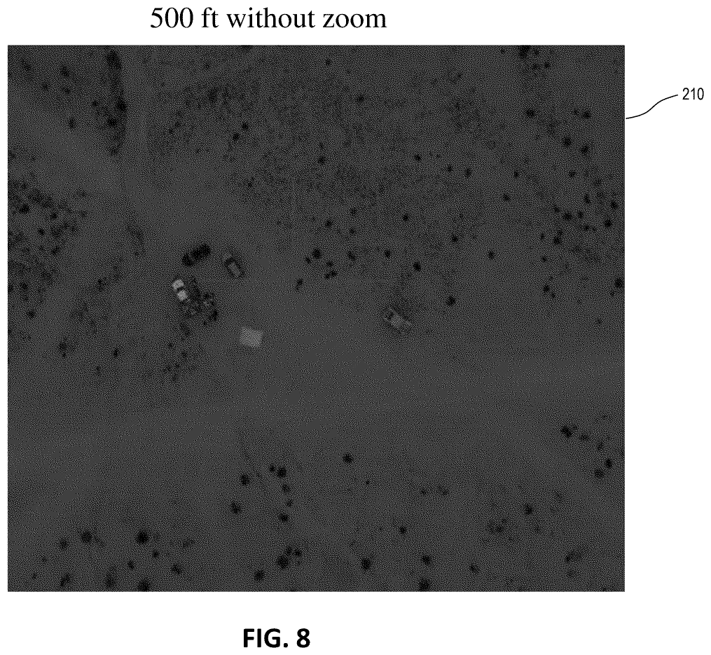

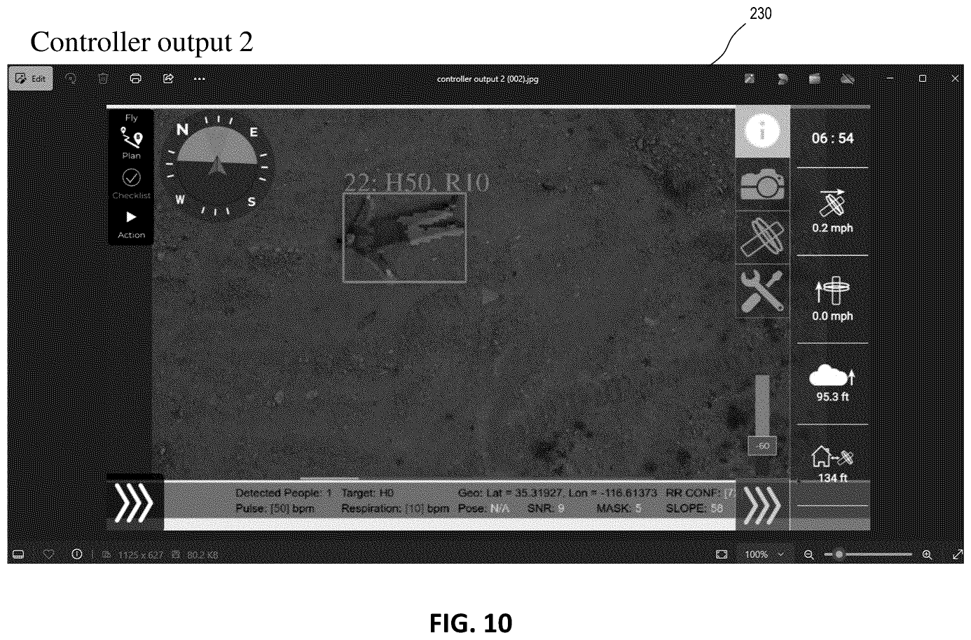

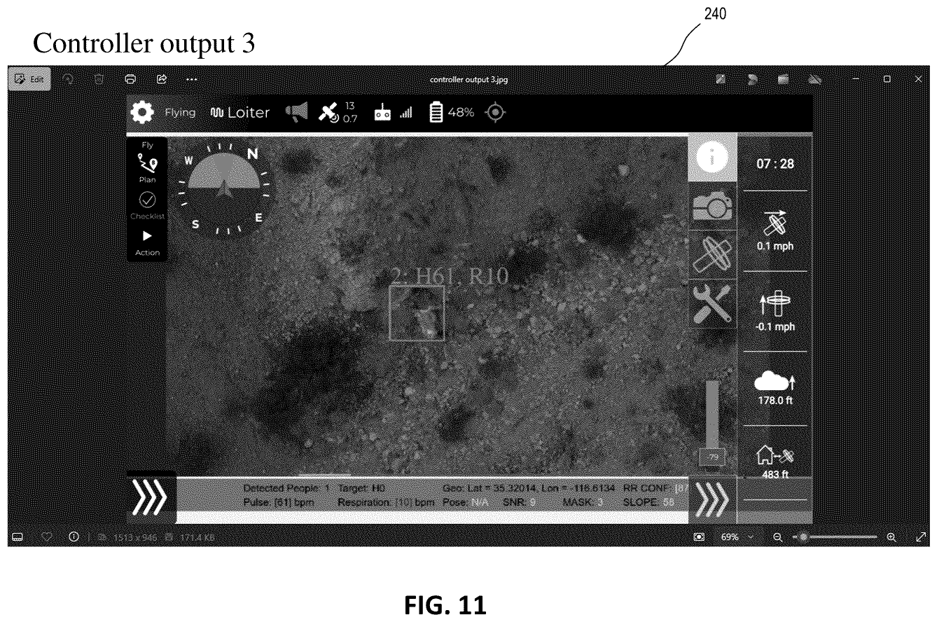

Referring now to FIGS. 7-11, different screen shots of the display 92 on the UAS controller 90 will be discussed. The screen shot 200 in FIG. 7 is a zoomed in view of a casualty 100 based on the UAS 30 at 500 feet in altitude. The vitals of the casualty 100 are provided. The screen shot 220 in FIG. 8 is the same casualty without zoom based on the UAS 30 at 500 feet in altitude. The UAS 30 does not perform detections while it is this zoomed out. The EO sensor 70 will zoom in to get a larger view of the casualty before the vison algorithms package 50 is executed by the processor 40.

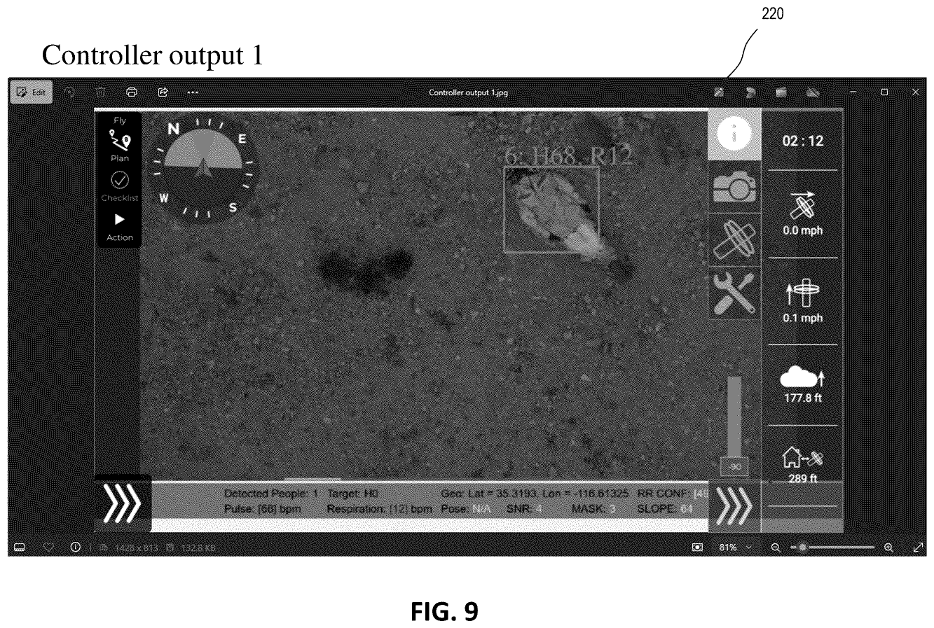

The screen shots 220, 230 and 240 in FIGS. 9-11 are different displays as provided on the UAS controller 90. The medic operating the UAS controller 90 is able to see the camera view of the system 30 as well as the onboard algorithms running in real-time for human detection and casualty assessment. The algorithms provide a bounding box around the casualty when detecting a human, and segments different detected body parts. The heart rate is shown as H.X and the respiration rate is shown as R.X in the image around the bounding box.

Another aspect is directed to a method for providing medical situational awareness 94 in a hazardous area 110 using the system 20 as described above. The method includes viewing the hazardous area 110 to provide EO sensor data 72, and executing a vision algorithm package 50 based on the EO sensor data to locate and analyze casualty victims within the hazardous area 110. The vision algorithm package 50 includes casualty detection algorithms 52 for casualty detection and identification; casualty assessment algorithms 54 for casualty respiration rate detection, pulse rate detection and motion detection; and gross injury detection algorithms 56 for casualty wound and hemorrhage detection, and kinematic irregularity detection. Control signals are received for control of the UAS 30. The casualty information as determined by the vision algorithm package 50 is transmitted to the UAS controller 90. The UAS controller 90 is used for providing the control signals to operate the UAS 30, and to display the live casualty information received from the UAS 30 to provide the medical situation awareness 94 for the hazardous area 110.

Many modifications and other embodiments will come to the mind of one skilled in the art having the benefit of the teachings presented in the foregoing descriptions and the associated drawings. Therefore, it is understood that the foregoing is not to be limited to the example embodiments, and that modifications and other embodiments are intended to be included within the scope of the appended claims.

1. A system for providing medical situational awareness in a hazardous area, comprising:
an unmanned aerial system (UAS) comprising:
a housing,
at least one electro-optical (EO) sensor carried by the housing and configured for viewing the hazardous area to provide EO sensor data,
a processor carried by the housing and coupled to the at least one EO sensor, and configured to execute a vision algorithm package based on the EO sensor data to locate and analyze casualty victims within the hazardous area, with the vision algorithm package comprising
casualty detection algorithms for casualty detection and identification,
casualty assessment algorithms for casualty respiration rate detection, pulse rate detection and motion detection, and
gross injury detection algorithms for casualty wound and hemorrhage detection, and kinematic irregularity detection,
a transceiver carried by the housing and coupled to the processor and configured to receive control signals and to transmit live casualty information as determined by the vision algorithm package; and
a UAS controller for providing the control signals to the UAS for control thereof, and comprising a display to display operator data for the UAS and the live casualty information received from the transceiver to provide the medical situation awareness for the hazardous area.

2. The system according to claim 1 wherein the UAS controller comprises a processor configured to aggregate the live casualty information over time, with the aggregated casualty information being transmitted to a network based on a confidence factor of the aggregated casualty information.

3. The system according to claim 2 comprising at least one mobile device in proximity to the casualty victims and configured to receive and display the aggregated casualty information from the network.

4. The system according to claim 1 wherein the vision algorithm package provides the casualty information in real-time to the UAS controller.

5. The system according to claim 1 wherein the casualty detection algorithms comprise a human detection algorithm, and a human identification algorithm.

6. The system according to claim 5 wherein the human detection algorithm is configured to detect individual body parts of casualty victims when partially occluded within the hazardous area

7. The system according to claim 1 wherein the casualty assessment algorithms comprise a respiration rate detection algorithm, a pulse rate detection algorithm and a motion detection algorithm.

8. The system according to claim 1 wherein the gross injury detection algorithms comprise a wound and hemorrhage detection algorithm, and a kinematic irregularity detection algorithm.

9. The system according to claim 1 wherein the UAS is configured as a small UAS (SUAS) weighing less than 55 pounds.

10. The system according to claim 1 wherein the at least one EO sensor comprises a camera.

11. The system according to claim 1 wherein the UAS comprises a global positioning system (GPS) configured to determine GPS coordinates of the casualty victims based on an angle trajectory of the camera.

12. A method for providing medical situational awareness in a hazardous area, comprising:
operating an unmanned aerial system (UAS) configured to perform the following:
view the hazardous area to provide EO sensor data,
execute a vision algorithm package based on the EO sensor data to locate and analyze casualty victims within the hazardous area, with the vision algorithm package comprising
casualty detection algorithms for casualty detection and identification,
casualty assessment algorithms for casualty respiration rate detection, pulse rate detection and motion detection,
gross injury detection algorithms for casualty wound and hemorrhage detection, and kinematic irregularity detection,
receive control signals for control of the UAS, and
transmit casualty information as determined by the vision algorithm package; and
using a UAS controller for providing the control signals to operate the UAS, and to display operator data for the UAS and to display the live casualty information received from the UAS to provide the medical situation awareness for the hazardous area.

13. The method according to claim 12 wherein the UAS controller comprises a processor configured to aggregate the live casualty information over time, with the aggregated casualty information being transmitted to a network based on a confidence factor of the aggregated casualty information.

14. The method according to claim 13 comprising a mobile device in proximity to the casualty victims and configured to receive and display the aggregated casualty information from the network.

15. The method according to claim 12 wherein the vision algorithm package provides the casualty information in real-time to the UAS controller.

16. The method according to claim 12 wherein the casualty detection algorithms comprise a human detection algorithm, and a human identification algorithm.

17. The method according to claim 16 wherein the human detection algorithm detects individual body parts of casualty victims when partially occluded within the hazardous area

18. The method according to claim 12 wherein the casualty assessment algorithms comprise a respiration rate detection algorithm, a pulse rate detection algorithm and a motion detection algorithm.

19. The method according to claim 12 wherein the gross injury detection algorithms comprise a wound and hemorrhage detection algorithm, and a kinematic irregularity detection algorithm.

20. The method according to claim 12 wherein the UAS is configured as a small UAS (SUAS) weighing less than 55 pounds.
